(19) United States
(12) Patent Application Publication
(10) Pub. No.: US 2024/0207416 A1
(43) Pub. Date: Jun. 27, 2024

(54) STEREOCOMPLEX OF OLIGOLACTIC ACID CONJUGATES IN MICELLES FOR IMPROVED PHYSICAL STABILITY AND ENHANCED ANTITUMOR EFFICACY

(71) Applicant: Wisconsin Alumni Research Foundation, Madison, WI (US)

(72) Inventors: Glen Kwon, Madison, WI (US); Yu Tong Tam, Madison, WI (US)

(73) Assignee: Wisconsin Alumni Research Foundation, Madison, WI (US)

(21) Appl. No.: 18/424,697

(22) Filed: Jan. 26, 2024

Related U.S. Application Data
(63) Continuation of application No. 17/044,189, filed on Sep. 30, 2020.
(60) Provisional application No. 62/651,365, filed on Apr. 2, 2018.

(51) Int. Cl. A61K 47/59; A61K 31/7068; A61K 47/69; A61P 35/04

(52) U.S. Cl. CPC A61K 47/593; A61K 31/7068; A61K 47/6907; A61P 35/04

(57) ABSTRACT

The present technology relates generally to oligolactic acid conjugates and stereocomplexes of conjugates of gemcitabine and gemcitabine derivatives, micelle compositions containing such conjugates or stereocomplexes of conjugates, and methods of preparing and using such compositions to treat various cancers. The oligolactic acid conjugates and stereocomplexes of conjugates may include oligolactic acid comprising 2 to 20 lactic acid subunits and may be attached through an amide linkage to the nitrogen of the 4(N) of the gemcitabine or gemcitabine derivative. Compositions comprising water and a micelle comprising a polylactic acid-containing polymer and the oligolactic acid conjugate or stereocomplex of conjugates may be readily prepared. Methods of inhibiting or killing cancer cells and treating gemcitabine sensitive cancers are also provided.

### CROSS-REFERENCE TO RELATED APPLICATIONS

This application claims the benefit of and priority to U.S. Provisional Patent Application No. 62/651,365, filed Apr. 2, 2018, the entire disclosure of which is hereby incorporated by reference in its entirety for any and all purposes.

### STATEMENT OF GOVERNMENT SUPPORT

This invention was made with government support under AI101157 awarded by the National Institutes of Health. The government has certain rights in the invention.

### TECHNICAL FIELD

The present technology relates generally to oligolactic acid conjugates and stereocomplexes of conjugates of gemcitabine and gemcitabine derivatives thereof. The conjugates and stereocomplexes of conjugates may be formulated in synthetic micelles to provide superior plasma stability and/or enhanced efficacy in the treatment of cancer compared to standard formulations of gemcitabine.

### BACKGROUND

Gemcitabine is a potent chemotherapeutic agent useful in the treatment of a variety of cancers and has the structure shown below.

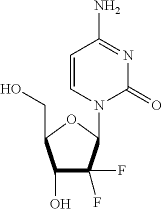

Gemcitabine is a highly water soluble nucleoside analogue designed to mimic the naturally occurring pyrimidine, cytidine. Once inside the cell, gemcitabine requires a series post-translational modifications to be active. However, gemcitabine is rapidly deaminated in blood plasma to an inactive metabolite 2′,2′-difluorodeoxyuridine and rapidly excreted through the urine. To increase the therapeutic levels in the blood, gemcitabine requires administration in high doses (˜1000 mg/m²) to overcome the short half-life.

### SUMMARY

The present technology provides a 4(N)-oligo-L-lactic acid conjugate of gemcitabine or a gemcitabine derivative; a 4(N)-oligo-D-lactic acid conjugate of gemcitabine or a gemcitabine derivative; and a stereocomplex of a 4(N)-oligo-L-lactic acid conjugate and a 4(N)-oligo-D-lactic acid conjugate. The conjugates or stereocomplex typically include 2 to 20 lactic acid subunits which may be attached through an amide linkage of the 4-amino group of the gemcitabine or gemcitabine derivative. In any embodiment, the conjugates in a stereocomplex may include 7 to 20 lactic acid subunits.

In some embodiments, the present technology provides conjugates and stereocomplexes of oligolactic acid and gemcitabine or gemcitabine derivatives having enhanced blood plasma stability and anti-cancer efficacy. The conjugates provided herein can be formulated into micelles as pharmaceutical compositions and medicaments that are useful in the treatment of cancer. Also provided is the use of the conjugates in preparing pharmaceutical formulations and medicaments.

### BRIEF DESCRIPTION OF THE DRAWINGS

FIG. 1 shows stereocomplex prodrugs of o(LLA)ₙ-GEM and o(DLA)ₙ-GEM for PEG-b-PLA micelles: loading, stability, and prodrug conversion by backbiting and esterase after release, according to an embodiment.

FIG. 2 shows the synthetic scheme for o(LLA)ₙ-GEM and o(DLA)ₙ-GEM, according to the examples.

FIG. 3A shows the powder XRD profiles of o(LLA)₁₀-GEM, o(DLA)₁₀-GEM and the stereocomplex mixture of both prodrugs after evaporation from CH₃CN, according to the examples. FIG. 3B shows the DSC thermograms of o(LLA)₁₀-GEM, o(DLA)₁₀-GEM and the stereocomplex mixture of both prodrugs after evaporation from CH₃CN, according to the examples. FIG. 3C shows the powder XRD profiles of o(LLA)₆-GEM, o(DLA)₆-GEM and the stereocomplex mixture of both prodrugs after evaporation from CH₃CN, according to the examples. FIG. 3D shows the DSC thermograms of o(LLA)₆-GEM, o(DLA)₆-GEM and the stereocomplex mixture of both prodrugs after evaporation from CH₃CN, according to the examples.

FIGS. 4A and 4B show AFM images of stereocomplex o(L+DLA)₁₀-GEM loaded PEG-b-PLA micelles, according to the examples.

FIG. 5 demonstrates the in vitro release of o(LLA)₆-GEM, o(LLA)₁₀-GEM or stereocomplex o(L+DLA)₁₀-GEM from PEG-b-PLA micelles at 5% and 15% loading (mean±SEM, n=3-4), according to the examples.

FIG. 6A shows the relative amount of the physical mixture of o(L+DLA)₁₀-GEM and its backbiting conversion products after incubation in 1:1 CH₃CN/10 mM PBS at 37° C., pH 7.4 from 0 to 72 hours (mean±SD, n=3), according to the examples. FIG. 6B shows the relative amount of o(L+DLA)₁₀-GEM loaded PEG-b-PLA micelles at 15% loading and its conversion product after incubation in MilliQ water at 25° C. from 0 to 8 weeks (mean±SD, n=3), according to the examples.

FIG. 7A shows the relative amount of GEM in rat plasma after incubation at 37° C. from 0 to 24 hours (mean±SEM, n=3), according to the examples. FIG. 7B shows the relative amount of o(L+DLA)₁₀-GEM in rat plasma after incubation at 37° C. from 0 to 24 hours (mean±SEM, n=3), according to the examples. FIG. 7C shows the relative amount of o(L+DLA)₁₀-GEM prodrugs in PEG-b-PLA micelles at 15% loading and its conversion product GEM in rat plasma after incubation at 37° C. from 0 to 24 hours (mean±SEM, n=3), according to the examples.

FIG. 8A shows in vitro cytotoxicity of GEM, and o(L+DLA)₁₀-GEM micelles at 15% loading against human A549 non-small lung cancer cells, according to the examples.

FIG. 8B shows in vitro cytotoxicity of GEM, and o(L+DLA)₁₀-GEM micelles at 15% loading against PANC-1 pancreatic cancer cells, according to the examples. Mean of quintuplicate determinations ±SEM.

FIGS. 9A and 9B show in vivo antitumor efficacies of GEM and o(L+DLA)₁₀-GEM micelles in an A549 non-small cell lung cancer xenograft model, according to the examples. Mice were administered I.V. weekly for three weeks (as indicated by the arrows) with GEM (10 mg/kg) or o(L+DLA)₁₀-GEM micelles (5% loading, 10 mg/kg GEM equivalent), according to the examples. FIG. 9A shows relative tumor volume (mean±SEM, n=3-4, *: p<0.05). FIG. 9B shows relative body weight (mean±SEM, n=3-4, *: p<0.05).

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The present technology provides pharmaceutical compositions and medicaments comprising any of one of the embodiments of the compounds (drugs and/or drug conjugates) and micelles disclosed herein and a pharmaceutically acceptable carrier or one or more excipients. The compositions may be used in the methods and treatments described herein. The pharmaceutical composition may include an effective amount of any of one of the embodiments of the compounds of the present technology disclosed herein. In any of the embodiments, the effective amount may be determined in relation to a subject. “Effective amount” refers to the amount of a compound, conjugate, micelle or composition required to produce a desired effect. One example of an effective amount includes amounts or dosages that yield acceptable toxicity and bioavailability levels for therapeutic (pharmaceutical) use including, but not limited to, the treatment of cancers or cardiovascular disease such as restenosis.

As used herein, a “subject” or “patient” is a mammal, such as a cat, dog, rodent or primate. Typically, the subject is a human, and, preferably, a human suffering from a cancer sensitive to gemcitabine or derivative thereof, i.e. a cancer capable of treatment with an effective amount of gemcitabine or derivative thereof. The term “subject” and “patient” can be used interchangeably.

As used herein, a “gemcitabine derivative” is a compound that retains the pyrimidine/nucleoside skeleton of gemcitabine but contains at least one modified side chain. Other gemcitabine derivatives are known to those of skill in the art and include, but are not limited to, cytarabine (AraC), emtricitabine, lamivudine, zalcitabine, azacytidine, and troxacitabine.

The term “stereocomplex” refers to a composition of stereoselectively associated pre-formed oligolactic acid conjugates, comprising a 4(N)-oligo-L-lactic acid conjugate of gemcitabine or a gemcitabine derivative and a 4(N)-oligo-D-lactic acid conjugate of gemcitabine or a gemcitabine derivative.

As used herein, a “hydroxyl protecting group” refers to —O-G groups. G is a hydroxyl protecting group. Hydroxyl protecting groups are well known to one of ordinary skill in the art. In some embodiments, the hydroxyl protecting group may be selected from the group consisting of: methoxymethyl ethers (MOM), methoxyethoxymethyl ethers (MEM), benzyloxymethyl ethers (BOM), tetrahydropyranyl ethers (THP), benzyl ethers (Bn), p-methoxybenzyl ethers, trimethylsilyl ethers (TMS), triethylsilyl ethers (TES), triisopropylsilyl ethers (TIPS), t-butyldimethylsilyl ethers (TBDMS), t-butyldiphenylsilyl ethers (TBDPS), o-nitrobenzyl ethers, p-nitrobenzyl ethers, trityl ethers, acetate, chloroacetate, dichloroacetate, trichloroacetate, trifluoroacetate, benzoate (Bz), methyl carbonate, allyl carbonate (alloc), dimethylthiocarbamate (DMTC), benzyl carbonate (Cbz), t-butyl carbonate (Boc), and 9-(fluorenylmethyl) carbonate (Fmoc). An extensive list of protecting groups for the hydroxyl group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

In one aspect, the present technology provides conjugates of oligo-L-lactic acid with gemcitabine and gemcitabine derivatives. In another aspect, the present technology provides conjugates of oligo-D-lactic acid with gemcitabine and gemcitabine derivatives. In another aspect, the present technology provides conjugates of stereocomplexes with gemcitabine and gemcitabine derivatives.

In the conjugates and stereocomplexes of the present technology, oligolactic acid may be a linear polyester of lactic acid. In the conjugates and stereocomplexes of the present technology, the oligolactic acid may be attached through an amide linkage to the nitrogen of the 4-amino of the gemcitabine or gemcitabine derivative. In such conjugates and stereocomplexes, the oligolactic acid typically includes 2 to 20 lactic acid subunits. It will be understood by those skilled in the art that the present conjugates and stereocomplexes may have 2, 3, 4, 5, 6, 7, 8, 9, 10, 11, 12, 13, 14, 15, 16, 17, 18, 19, or 20 lactic acid subunits or a range of subunits between any two of the foregoing values. For example, the conjugates and stereocomplexes may include 7 to 20 lactic acid subunits. In any embodiment, the oligo-L-lactic acid and oligo-D-lactic acid may include at least 2, at least 3, at least 4, at least 5, at least 6, or at least 7 lactic acid subunits each.

In any embodiment, the conjugates and stereocomplexes may have the structure shown in formula I:

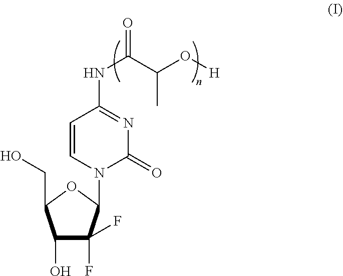

wherein n at each occurrence is individually an integer from 2 to 20 or a range between including endpoints (e.g., 2, 3, 4, 5, 6, 7, 8, 9, 10, 11, 12, 13, 14, 15, 16, 17, 18, 19, or 20). In any embodiment, n at each occurrence may be an integer from 7 to 20 or a range between including endpoints. In some embodiments, the oligolactic acid may be L-oligolactic acid. In some embodiments, the oligolactic acid may be D-oligolactic acid.

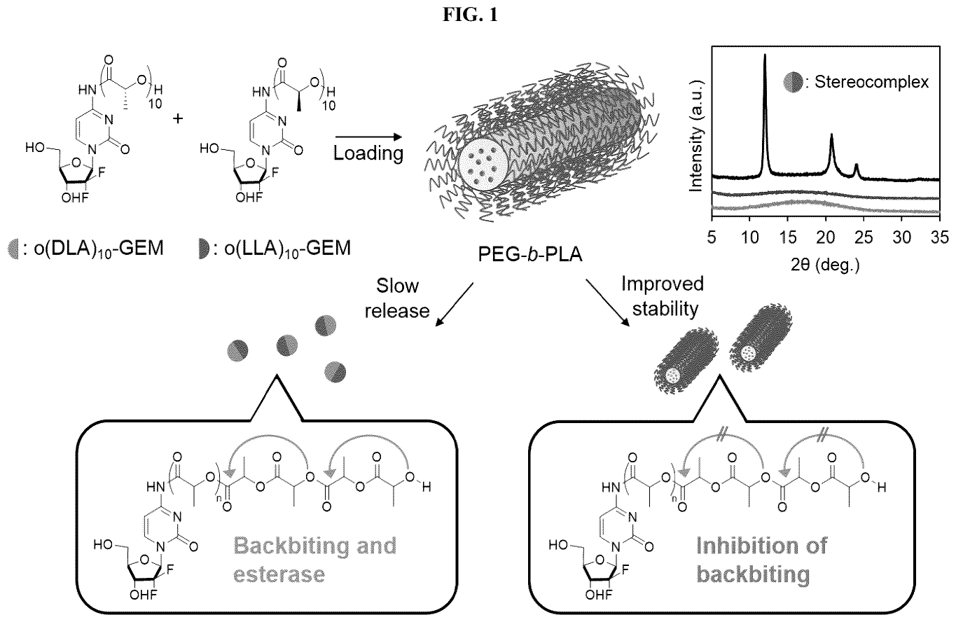

The present technology provides compositions comprising a stereocomplex of a 4(N)-oligo-L-lactic acid conjugate of gemcitabine or a gemcitabine derivative and a 4(N)-oligo-D-lactic acid conjugate of gemcitabine or a gemcitabine derivative. As indicated above, the oligo-L-lactic acid or the oligo-D-lactic acid in each conjugate may include 2 to 20 lactic acid subunits and/or may be attached through an amide linkage to the nitrogen of the 4-amino of the gemcitabine or gemcitabine derivative. The length of the oligolactic acid may vary as indicated above, e.g., the oligolactic acid may have from 7 to 20 lactic acid residues. In the stereocomplex, the molar ratio of the two conjugates may be about 1:1. However, compositions comprising the stereocomplex may include a molar ratio of the 4(N)-oligo-L-lactic acid conjugate of gemcitabine or a gemcitabine derivative to the 4(N)-oligo-D-lactic acid conjugate of gemcitabine or a gemcitabine derivative that ranges from 2:1 to 1:2, e.g., 3:2 or 2:3 or 1:1. FIG. 1 illustrates schematically for one embodiment of the present technology the stereocomplexes, o(L+DLA)ₙ-GEM, and their loading into micelles.

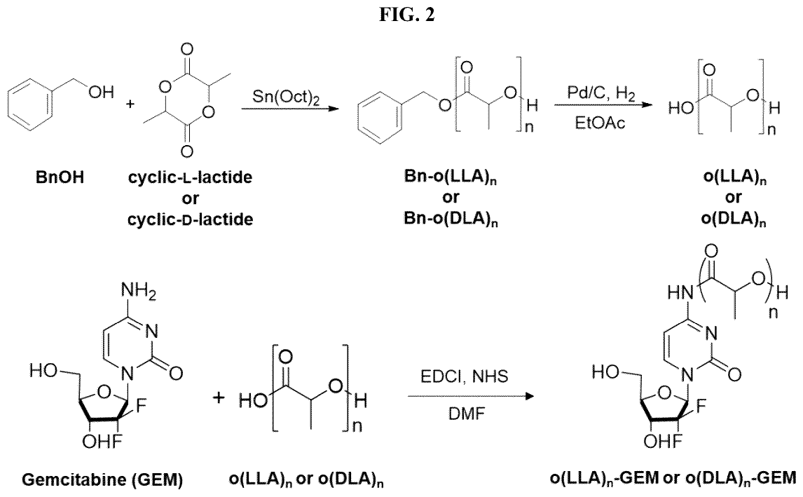

In any embodiment, the conjugates of the present technology may be prepared by contacting gemcitabine or a gemcitabine derivative having a free 4-amino group with a coupling agent and oligolactic acid having 2 to 20 lactic acid subunits and a hydroxyl. In any embodiment, the hydroxyl may be a protected hydroxyl group or previously a protected hydroxyl group. In any embodiment, the hydroxyl group may be deprotected prior to the contacting with the gemcitabine or the gemcitabine derivative. In any embodiment, the protected hydroxyl group may include a benzyl protecting group. In any embodiment, prior to the hydroxyl group being deprotected, the oligolactic acid may be a benzyl-oligolactic acid having 2 to 20 lactic acid subunits. By way of example only, FIG. 2 provides an illustrative embodiment of the method of making the present conjugates. In FIG. 2, gemcitabine is coupled to a carboxyl on L- or D-oligolactic acid intermediate using a coupling reagent in a suitable organic solvent. Suitable coupling agents include carbodiimides such as DCC and EDCI. Suitable organic solvents include halogenated solvents (e.g., dichloromethane, chloroform), alkyl acetate (e.g., ethyl acetate), or other polar aprotic solvent (e.g., DMF, THF).

In another aspect, the present technology provides aqueous compositions of micelles formed from water, polylactic acid-containing polymers and any gemcitabine/gemcitabine derivative oligolactic acid-conjugates of the present technology described herein. In another aspect, the present technology provides aqueous compositions of micelles formed from water, polylactic acid-containing polymers and any stereocomplexes of gemcitabine/gemcitabine derivative oligolactic acid-conjugates of the present technology described herein.

In any embodiment, the micelles may include the block copolymer, PEG-b-PLA (also known as PEG-PLA). The poly(lactic acid) block may include (D-lactic acid), (L-lactic acid), (D,L-lactic acid), or combinations thereof. Various forms of PEG-b-PLA are available commercially, such as from Polymer Source, Inc., Montreal, Quebec, or they can be prepared according to methods well known to those of skill in the art. In any embodiment, the molecular weight of the poly(ethylene glycol) block (PEG block) may be about 1,000 to about 35,000 g/mol, or any increment of about 500 g/mol within said range. (Unless otherwise specified all polymer molecular weights referred to herein will be understood to be weight average molecular weights.) For example, the molecular weight of the PEG block may be about 1,000, 1,500, 2,000, 2,500, 3,000, 3,500, 4,000, 4,500, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000 or a range between and including any two of the foregoing values. In any embodiment, suitable blocks of the poly(lactic acid) block (PLA block) may have molecular weights of about 1,000 to about 15,000 g/mol, or any increment of about 500 g/mol within said range. For example, the molecular weight of the PLA block may be about 1,000, 1,500, 2,000, 2,500, 3,000, 3,500, 4,000, 4,500, 5,000, 6,000, 6,500, 7,000, 7,5000, 8,000, 8,500, 9,000, 9,500, 10,000, 10,500, 11,000, 11,500, 12,000, 12,500, 13,000, 13,500, 14,000, 14,500, 15,000, or a range between and including any two of the foregoing values. In any embodiment, the PEG block may terminate in an alkyl group, such as a methyl group (e.g., a methoxy ether) or any suitable protecting, capping, or blocking group. In any embodiment, the molecular weight of the PEG block may be about 1,000 to about 35,000 g/mol, the molecular weight of the PLA block may be about 1,000 to about 15,000 g/mol, or a combination thereof. In any embodiment, the molecular weight of the PEG block may be about 1,500 to about 14,000 g/mol, the molecular weight of the PLA block may be about 1,500 to about 7,000 g/mol, or a combination thereof.

In any embodiment, the micelles of the present technology may be prepared using PEG-b-PLA polymers of a variety of block sizes (e.g., a block size within a range described above) and in a variety of ratios. For example, the PEG:PLA ratio may be about 1:10 to about 10:1, or any integer ratio within said range, including without limitation 1:5, 1:3, 1:2, 1:1, 2:1, 3:1, and 5:1. For example, number average molecular weights (Mₐ) of the PEG-PLA polymers can include, but are not limited to, about 2K-2K, 3K-5K, 5K-3K, 5K-6K, 6K-5K, 6K-6K, 8K-4K, 4K-8K, 12K-3K, 3K-12K, 12K-6K, 6K-12K (PEG-PLA, respectively) or a range between and including any two of the foregoing values.

One suitable PEG-PLA polymer includes blocks of about 1-3 kDa (e.g., about 2K Daltons) at an approximate 1:1 ratio. Use of this block polymer resulted in high levels of drug-conjugate loading in the micelles. Further specific examples of PEG-PLA molecular weights include 4.2K-b-1.9K; 5K-b-10K; 12K-b-6K; 2K-b-1.8K, and those described in the Examples below. Other suitable amphiphilic block copolymers that may be used are described in U.S. Pat. No. 4,745,160 (Churchill et al.) and 6,322,805 (Kim et al.), each of which is herein incorporated by reference. The drug-to-polymer ratio may be about 1:20 to about 2:1, or any integer ratio within said range. Specific examples of suitable drug-polymer ratios include, but are not limited to, about 2:1, about 3:2, about 1.2:1, about 1:1, about 3:5, about 2:5, about 1:2, about 1:5; about 1:7.5; about 1:10, about 1:20 or a range between and including any of the foregoing values.

The micelles of the present technology may be loaded with a wide range of amounts, including high amounts, of the conjugates and stereocomplexes described herein. For example, the loading of the conjugates and stereocomplexes may be from about 1 wt % to about 50 wt % with respect to the mass of the micelles. Examples of conjugate and stereocomplex loading in the micelles include about 1 wt %, about 2 wt %, about 3 wt %, about 4 wt %, about 5 wt %, about 10 wt %, about 15 wt %, about 20 wt %, about 25 wt %, about 30 wt %, about 35 wt %, about 40 wt %, about 45 wt %, or about 50 wt % with respect to the mass of the micelles, or a range between and including any two of the foregoing values. In any embodiment, the loading of the conjugates and stereocomplexes may be from about 5 wt % to about 15 wt %.

Loading of each conjugate and stereocomplex of conjugates in the micelles may also be expressed in terms of concentration. For example, the concentration of each conjugate and stereocomplex of conjugates may be from about 0.5 mg/mL to about 40 mg/mL with respect to the volume of the water in the composition. Examples of each conjugate concentration that may be obtained with the present technology include about 0.6 mg/mL, about 1 mg/mL, about 2 mg/mL, about 3 mg/mL, about 4 mg/mL, about 5 mg/mL, about 6 mg/mL, about 8 mg/mL, about 10 mg/mL, about 12 mg/mL, about 15 mg/mL, about 20 mg/mL, about 25 mg/mL, about 30 mg/mL, about 35 mg/mL, or about 40 mg/mL with respect to the volume of the water in the composition, or a range between and including any two of the foregoing values. In any embodiment, the concentration of the L-oligolactic acid conjugate may be about 1 to about 15 mg/mL or even about 2 to about 12 mg/mL, the concentration of the D-oligolactic acid conjugate may be about 1 to about 20 mg/mL or even about 1.5 to about 10 mg/mL, and/or the concentration of the stereocomplex of conjugates may be about 0.5 to about 15 mg/mL or even about 1 to about 10 mg/mL.

The loading of each conjugate and stereocomplex in the micelles may also be expressed in terms of loading efficiency. For example, the loading efficiency of each conjugate and stereocomplex may be from about 25 wt % to about 100 wt % with respect to the mass of the micelles. Examples of conjugate and stereocomplex of conjugates loading efficiency in the micelles include about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 99 wt %, or about 100 wt % with respect to the mass of the micelles, or a range between and including any two of the foregoing values. In any embodiment, the loading efficiency of the L-oligolactic acid conjugate may be at least about 50 wt % including about 80 wt % to about 90 wt % and the loading efficiency of the stereocomplex may be at least about 50 wt % including from about 90 wt % to about 100 wt %.

In any embodiment, the present technology provides compositions comprising water and a micelle including PEG-b-PLA and at least one of the L-oligolactic acid conjugates, the D-oligolactic acid conjugates, and the stereocomplexes described herein. In any embodiment, the loading of the loading of the L-oligolactic acid conjugates may be from about 1 wt % to about 50 wt %; the loading of the D-oligolactic acid conjugates may be from about 1 wt % to about 50 wt %; and/or the loading of the stereocomplexes may be from about 1 wt % to about 50 wt % with respect to the mass of the micelles. In any embodiment, the molecular weight of the PEG block of the PEG-b-PLA may be about 1,500 to about 14,000 g/mol; the molecular weight of the PLA block of the PEG-b-PLA may be about 1,500 to about 7,000 g/mol, or a combination thereof. Such compositions may include any of the gemcitabine loadings described herein, including e.g., about 1 wt % to about 50 wt %, about 1 to about 15 mg/mL, or about 2 to about 12 mg/mL of any of the L-oligolactic acid conjugates; about 5 wt % to about 50 wt %, about 1 to about 20 mg/mL, or about 2 to about 10 mg/mL of any of the D-oligolactic acid conjugates; and/or about 2 wt % to about 30 wt %, about 1 to about 15 mg/mL, or about 2 to about 15 mg/mL of any of the stereocomplexes. In any embodiment, the composition may include any of the L-oligolactic acid conjugates, any of the D-oligolactic acid conjugates, any of the stereocomplexes as described herein, or a combination of two or more thereof.

Amphiphilic single chains of amphiphilic polymers present in a solvent in an amount above the critical micelle concentration (CMC) aggregate into a micelle, a core-coronal structure with a hydrophobic interior, and hydrophilic exterior or shell. Proton NMR spectroscopic studies of drug or conjugate loaded PEG-b-PLA micelles indicate that while the micelles readily form in aqueous environments, they decompose in organic solvents such as DMSO. The present micelle compositions typically are substantially free of organic solvents, e.g., less than about 2 wt % of ethanol, dimethyl sulfoxide, castor oil, and castor oil derivatives (i.e., polyethoxylated camphor compounds such as Cremophor EL) based on the weight of the composition. In any embodiment, the amount of organic solvent may be less than about 1 wt %, less than about 0.5 wt %, less than about 0.1 wt %, or essentially free of detectable amounts of organic solvents.

PEG-b-PLA micelles may be prepared as described below in this section, as well as below in the Examples. The composition of micelles described herein may be prepared by combining water with a mixture of a polylactic acid-containing polymer and the drug/drug derivative combination of gemcitabine. In another embodiment, the composition of micelles described herein may be prepared by combining water with a mixture of a polylactic acid-containing polymer and at least one of the drug/drug derivative conjugates/stereocomplex of conjugates described herein. In any embodiment, the polylactic acid-containing polymer may include PEG-b-PLA.

The procedures given below are merely illustrative. The procedure can be varied according to the desired scale of preparation, as would be readily recognized by one skilled in the art. One advantage of the procedure is that it does not require the dialysis of a micelle solution.

Preparatory Procedure: Freeze-drying. In one embodiment, micelle preparation can be carried out as follows. At least one conjugate or stereocomplex as described herein loaded in a PEG-b-PLA micelle can be prepared by freeze-drying from a tert-butanol-water mixture. For example, 2-20 mg of PEG4000-b-PLA2200 (Advanced Polymer Materials Inc., Montreal, Canada) and 1.0 mg of a conjugate(s) as described herein can be dissolved in 1.0 mL of tert-butanol at 60° C., followed by addition of 1.0 mL of pre-warmed double-distilled water at 60° C. with vigorous mixing. The mixture is allowed to freeze in dry ice/ethanol cooling bath at −70° C. Lyophilization may then be performed on a shelf freeze-dryer at −20° C. shelf inlet temperature for 72 h at 100 pBar throughout the experiment. The lyophilized cake may then rehydrated with 1.0 mL of 0.9% saline solution at 60° C., centrifuged, filtered through 0.22 m regenerated cellulose filter, and analyzed by HPLC. In some embodiments, this procedure is used to prepare micelles of oligo-L-lactic acid conjugates or oligo-D-lactic acid conjugates. In some embodiments, this procedure is used to prepare micelles of stereocomplexes.

Once prepared, the micelle-conjugate or micelle-stereocomplex compositions can be stored for extended periods of time under refrigeration, preferably at a temperature below about 5° C. Temperatures between about −20° C. and about 4° C. have been found to be suitable conditions for storage of most micelle-conjugate and micelle-stereocomplex compositions. For example, aqueous solutions of the present conjugate-loaded micelles may be stored at about 4° C. Freeze-dried micelle compositions as described herein can be stored at −20° C. for prolonged periods and then rehydrated. Use of brown glass vials or other opaque containers to protect the micelle compositions from light can further extend effective lifetimes of the compositions.

In any embodiment, the present technology provides methods of inhibiting or killing cancer cells sensitive to gemcitabine or a gemcitabine derivative. In any embodiment the method may include contacting the cells with an effective inhibitory or lethal amount of any of the compositions described herein. In any embodiment, the contacting may be performed in vitro or in vivo. There are also provided methods of treatment including administering to a mammal suffering from a cancer sensitive to gemcitabine or a gemcitabine derivative, an effective amount of the micelle compositions described herein. Examples of gemcitabine-sensitive cancers include ovarian cancer, leukemia, angiosarcoma, breast cancer, colorectal cancer, prostate cancer, lung cancer, pancreatic cancer, cholangiocarcinoma, brain cancer (such as gliomas), adenocarcinomas, hepatomas, and biliary tract cancer. In any embodiment, the cancer may be lung cancer or pancreatic cancer.

In any of the embodiments of the present technology described herein, the pharmaceutical composition may be packaged in unit dosage form. The unit dosage form is effective in treating a cancer. Generally, a unit dosage including a composition of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

Micelle compositions containing conjugates/stereocomplexes of gemcitabine or gemcitabine derivatives may be prepared as described herein and used to treat cancers and cardiovascular diseases. The conjugates, stereocomplexes and compositions described herein may be used to prepare formulations and medicaments that treat restenosis or a cancer, such as ovarian cancer, leukemia, angiosarcoma, breast cancer, colorectal cancer, prostate cancer, lung cancer, pancreatic cancer, cholangiocarcinoma, brain cancer (such as gliomas), adenocarcinomas, hepatomas, or biliary tract cancer. Such compositions can be in the form of, for example, granules, powders, tablets, capsules, syrup, suppositories, injections, emulsions, elixirs, suspensions or solutions. The instant compositions can be formulated for various routes of administration, for example, by parenteral, rectal, nasal, vaginal administration, or via implanted matrix or reservoir, or for restenosis, by drug-coated stent or balloon-based delivery. Parenteral or systemic administration includes, but is not limited to, subcutaneous, intravenous, intraperitoneal, and intramuscular, injections. The following dosage forms are given by way of example and should not be construed as limiting the instant present technology.

Injectable dosage forms generally include solutions or aqueous suspensions or oil in water suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution.

For injection, the pharmaceutical formulation and/or medicament may be a film or powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. In any embodiment, the injectable formulations include an isotonicity agent (e.g., NaCl and/or dextrose), buffer (e.g., phosphate) and/or a preservative.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

Specific dosages may be adjusted depending on conditions of disease, the age, body weight, general health conditions, sex, and diet of the subject, dose intervals, administration routes, excretion rate, and combinations of free drugs/conjugates. Any of the above dosage forms containing effective amounts are well within the bounds of routine experimentation and therefore, well within the scope of the instant present technology. By way of example only, such dosages may be used to administer effective amounts of the free drugs/conjugates to the patient and may include about 10 mg/m², about 20 mg/m², about 30 mg/m², about 40 mg/m², about 50 mg/m², about 75 mg/m², about 100 mg/m², about 125 mg/m², about 150 mg/m², about 200 mg/m², about 250 mg/m², about 300 mg/m², or a range between and including any two of the forgoing values. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In a related embodiment, method for treating a subject is provided, where the method involves administration of any one of the embodiments of the compositions of the present technology to a subject suffering from a cancer or a cardiovascular disease. In the method, it may be that the cancer is ovarian cancer, leukemia, angiosarcoma, breast cancer, colorectal cancer, prostate cancer, lung cancer, pancreatic cancer, cholangiocarcinoma, brain cancer (such as gliomas), adenocarcinomas, hepatomas, or biliary tract cancer.

In any of the embodiments of the method, the method may involve administration of a pharmaceutical composition, where the pharmaceutical composition includes any one of the embodiments of the conjugates or micelles containing the free drugs or conjugates of the present technology as well as a pharmaceutically acceptable carrier.

The examples herein are provided to illustrate advantages of the present technology and to further assist a person of ordinary skill in the art with preparing or using the conjugates and micelle compositions of the present technology, pharmaceutical compositions, derivatives, metabolites, prodrugs, racemic mixtures or tautomeric forms thereof. To the extent that the free drugs/conjugates/stereocomplexes include free drugs/conjugates/stereocomplexes of ionizable gemcitabine or derivatives thereof, salts such as pharmaceutically acceptable salts may also be used. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

The present technology is further illustrated by the following Examples, which should not be construed as limiting in any way.

### Example 1: Experimental Materials and Methods

Materials. All chemicals were purchased from Sigma-Aldrich (St. Louis, MO) and used as received. Analytical grade organic solvents and all other reagents were purchased from Fisher Scientific (Pittsburgh, PA). Gemcitabine (GEM) was purchased from LC Laboratories (Woburn, MA). PEG-b-PLA was purchased from Advanced Polymer Materials Inc. (Montreal, Canada): Mₙ of PEG and PLA was 4,000 and 2,200 g/mol, respectively; PDI 1.05. A549 human lung adenocarcinoma cells and PANC-1 pancreatic adenocarcinoma cells were purchased from ATCC (Manassas, VA) and cultured in RPMI 1640 medium and DMEM, respectively, supplemented with 10% fetal bovine serum (FBS), 100 IU/mL penicillin, 100 μg/mL streptomycin, and 2 mM_{L}-glutamine in 5% CO₂ incubator at 37° C. Heparinized Sprague-Dawley rat plasma and human pool plasma were purchased from Innovative Research Inc. (Novi, MI).

Instrumentation. Proton nuclear magnetic resonance (¹H NMR) data were recorded on a Varian Unity-Inova two-channel 500 MHz NMR spectrometer (Palo Alto, CA) with regulated temperature at 25° C. Chemical shifts (6) were reported in parts per million (ppm) relative to residual protonated solvent resonance at 7.26 ppm for CDCl₃. Mass spectrometry data was obtained using the Waters LCT (ESI-TOF) (Waters Corp., Milford, MA) in the Chemical Instrumentation Center in the Department of Chemistry, University of Wisconsin-Madison. Samples were sprayed from a 10 mM NH₄OAc/CH₃CN solution.

Reverse-phase HPLC (RP-HPLC) analysis was carried out using a Shimadzu Prominence HPLC system (Shimadzu, Kyoto, Japan) equipped with an LC-20AT pump, a SIL-20AC HT autosampler, a CTO-20AC column oven, and a SPD-M20A diode array detector. Sample was separated by a Waters Symmetry Shield™ RP₁₈ column (4.6 mm×250 mm, 5 μm, 100 Å). In a typical experiment, 10 μL of sample was injected at a flow rate of 0.8 mL/min, column temperature at 25° C., and UV detection wavelength at 270 nm for GEM and 300 nm for o(LA)ₙ-GEM. The separation of o(LA)ₙ-GEM conversion products was done in gradient mode with organic phase containing 100% CH₃CN as solvent A, and aqueous phase containing 100% milliQ water containing 0.1% formic acid as solvent B. Gradient elution was employed as follows: 0 min, 20% solvent A and 80% solvent B; 30 min, 68% solvent A and 32% solvent B; and 35 min for equilibration. Hydrodynamic diameters of PEG-b-PLA micelles were measured by dynamic light scattering (DLS) using a Zetasizer Nano-ZS (Malvern Instruments Inc., Malvern, England) at 25° C. with a detection angle of 173° and a He—Ne ion laser as the light source (4 mW, 633 nm). Prior to measurements, PEG-b-PLA micelle solutions were diluted with milliQ water or PBS (10 mM, pH 7.4) to afford the level of PEG-b-PLA at ˜ 0.1 mg/mL and 1 mL of each sample was placed into a disposable sizing cuvette (BrandTech Scientific Inc., Essex, CT). The cumulant method was used to curve-fit the correlation function, and the z-average diameter and polydispersity index (PDI) of PEG-b-PLA micelles were calculated from the Stokes-Einstein equation and the slope of the correlation function, respectively. All measurements were performed in triplicate. The morphology of stereocomplex o(L+DLA)₁₀-GEM loaded PEG-b-PLA micelles were observed using an atomic force microscope (AFM) in AC mode after adsorption of the polymer at 50.0 mg/mL on mica. Micelles were imaged in MilliQ water using an AC40 biolever on an Infinity Bioscope (Asylum Research, Santa Barbara, CA).

Synthesis of monodisperse benzyl-oligo(L-lactic acid)ₙ (Bn-o(LLA)ₙ) or benzyl-oligo(D-lactic acid)ₙ (Bn-o(DLA)ₙ). Synthesis of polydisperse was initiated with tin(II)-ethylhexanoate (Sn(Oct)₂) according to previously reported procedure with modifications (see De Jong, S. J. et al., Macromolecules, 1998, 31(19):6397-6402). For example, at an average degree of polymerization of 8, cyclic L-lactide or cyclic D-lactide was mixed with benzyl alcohol in a molar ratio of 4 to 1. The mixture was stirred at 130° C. until molten. Subsequently, 0.01 eq. of Sn(Oct)₂ in toluene (100 mg/mL) was added. The mixture was stirred at 130° C. for 4 hours and allowed to cool to room temperature, to obtain polydisperse Bn-o(LLA)ₙ or Bn-o(DLA)ₙ. Monodisperse Bn-o(LLA)₆, Bn-o(LLA)₁₀, Bn-o(DLA)₆, or Bn-o(DLA)₁₀ was fractionated via a CombiFlash Rf 4× system using C₁₈ reverse phase column chromatography. Gradient elution of acetonitrile in 0.1% formic acid and water in 0.1% formic acid was applied. The purified product was concentrated under reduced pressure to provide a colorless liquid. (Yield: 8.3% for Bn-o(LLA-)₆, 8.0% for Bn-o(DLA)₆, 6.7% for Bn-o(LLA)₁₀, and 7.9% for Bn-o(DLA)₁₀). ESI-TOF of Bn-o(LLA)₆: m/z calcd C₂₅H₃₂O₁₃ [M+Na]⁺: 558.2181, found 558.2179; ESI-TOF of Bn-o(DLA)₆: m/z calcd C₂₅H₃₂O₁₃ [M+Na]⁺: 558.2181, found 558.2178; ESI-TOF of Bn-o(LLA)₁₀: m/z calcd C₃₇H₄₈O₂₁ [M+Na]⁺: 846.3026, found 846.3027; ESI-TOF of Bn-o(DLA)₁₀: m/z calcd C₃₇H₄₈O₂₁ [M+Na]⁺: 846.3026, found 846.3023.

Synthesis of oligo(L-lactic acid)ₙ-gemcitabine (o(LLA)ₙ-GEM) or oligo(D-lactic acid)ₙ-gemcitabine (o(DLA)ₙ-GEM). o(LLA)ₙ-GEM or o(DLA)ₙ-GEM was prepared by amidation of a carboxylic acid group on o(LLA)ₙ or o(DLA)ₙ and an amine group on a gemcitabine molecule. In general, direct hydrogenation of Bn-o(LLA)ₙ or Bn-o(DLA)ₙ was achieved over palladium on activated carbon (10 wt %) to afford o(LLA)ₙ or o(DLA)ₙ according to a previously published report (Yield: −90-99%, see Takizawa, K. et al., J. Polym. Sci. A Polym. Chem., 2008, 46:5977-5990). Subsequently, 1-ethyl-3-(3-dimethylaminopropyl)carbodiimide (EDCI) (1.5 eq.) and N-hydroxysuccinimide (NHS) (1.5 eq.) were added to a solution of o(LLA)ₙ or o(DLA)ₙ(1 eq.) in dry DMF and stirred at room temperature under argon for 2 hours. GEM (1 eq.) was dissolved in dry DMF and was added dropwise into the above mixture. The reaction mixture was stirred for 24 hours at room temperature under argon. The crude products were diluted with ethyl acetate (20-fold), washed with 0.5% w/v hydrochloric acid, saturated NaHCO₃, and brine solution. The organic layer was then collected, dried over MgSO₄ and filtered. Solvent was removed under reduced pressure and the resulting concentrate was purified via a CombiFlash Rf 4× system using gradient elution of hexane and ethyl acetate. The purified product was concentrated under reduced pressure to provide a white solid (Yield: 80% for o(LLA)₆-GEM, 63% for o(DLA)₆-GEM, 87% for o(LLA)₁₀-GEM, and 74% for o(DLA)₁₀-GEM). ¹H NMR of o(LLA)₆-GEM (CDCl₃,500 MHz): δ=8.09 (d, J=7.6 Hz, 1H), 7.36 (br. s., 1H), 6.20 (br. s., 1H), 5.30 (s, 1H), 5.14-5.29 (m, 5H), 4.45-4.57 (m, 1H), 4.37 (q, J=7.1 Hz, 1H), 4.00-4.09 (m, 2H), 3.92 (d, J=10.5 Hz, 1H), 1.46-1.62 ppm (m, 18H). ESI-TOF: m/z calcd C₂₇H₃₅F₂N₃O₁₆ [M+H]⁺: 696.2058, found 696.2061; ¹H NMR of o(DLA)₆-GEM (CDCl₃, 500 MHz): δ=8.10 (d, J=7.3 Hz, 1H), 7.40 (br. s., 1H), 6.17 (br. s., 1H), 5.34 (d, J=7.1 Hz, 1H), 5.10-5.31 (m, 5H), 4.58 (br. s., 1H), 4.37 (q, J=6.8 Hz, 1H), 3.99-4.09 (m, 2H), 3.92 (d, J=10.7 Hz, 1H), 1.47-1.61 ppm (m, 18H). ESI-TOF: m/z calcd C₂₇H₃₅F₂N₃O₁₆ [M+H]⁺: 696.2058, found 696.2058; ¹H NMR of o(LLA)₁₀-GEM (CDCl₃,500 MHz): δ=8.08 (d, J=7.3 Hz, 1H), 7.39 (d, J=5.9 Hz, 1H), 6.19 (br. s., 1H), 5.13-5.33 (m, 10H), 4.54 (br. s., 1H), 4.37 (q, J=6.8 Hz, 1H), 4.05 (d, J=10.3 Hz, 2H), 3.93 (d, J=10.0 Hz, 1H), 1.46-1.63 ppm (m, 30H). ESI-TOF: m/z calcd C₃₉H₅₁F₂N₃O₂₄ [M+H]⁺: 984.2903, found 984.2907; ¹H NMR of o(DLA)₁₀-GEM (CDCl₃,500 MHz): δ=8.04 (d, J=8.1 Hz, 1H), 7.32-7.49 (m, 1H), 6.18 (br. s., 1H), 5.11-5.41 (m, 10H), 4.56-4.70 (m, 1H), 4.36 (d, J=6.3 Hz, 1H), 4.02-4.09 (m, 2H), 3.93 (d, J=10.0 Hz, 1H), 1.48-1.65 ppm (m, 30H). ESI-TOF: m/z calcd C₃₉H₅₁F₂N₃O₂₄ [M+H]⁺: 984.2903, found 984.2905.

Formation and characterization of stereocomplex o(LLA)₁₀-GEM and o(DLA)₁₀-GEM. Differential Scanning Calorimetry (DSC) measurements were performed in crimped aluminum pans with approximately 5-10 mg materials, using a TA Q2000 differential scanning calorimeter. The samples were cooled and heated at 10 K/min under 50 ml/min N₂ purge. Powder X-ray diffraction (PXRD) pattern were collected at room temperature from 2 to 400 2θ with a step size of 0.02° and an integration time of 1 s, using a Bruker D8 Advance diffractometer (Bruker, Billerica, MA). To prepare stereocomplex of o(LLA)₁₀-GEM and o(DLA)₁₀-GEM, equal amount of o(LLA)₁₀-GEM (4 mg) and o(DLA)₁₀-GEM (4 mg) were first dissolved in 100 μL of acetonitrile in a glass vial and swirled for 30 seconds. The solution was then placed on the DSC substrates or the silicon wafer and dried in vacuum at 70° C. for 12 h to yield the solid.

Preparation and characterization of PEG-b-PLA micelles containing stereocomplex o(L+DLA)₁₀-GEM, o(LLA)₁₀-GEM, or o(LLA)₆-GEM. Stereocomplex o(L+DLA)₁₀-GEM, o(LLA)₁₀-GEM, or o(LLA)₆-GEM loaded PEG-b-PLA micelles were prepared freeze-drying from a tert-butanol-water mixture (see Fournier, E. et al., Pharm. Res., 2004, 21(6):962-968). In a typical experiment, 1.0 mg of o(LLA)₁₀-GEM and 1.0 mg of o(DLA)₁₀-GEM, and 10.0 mg or 20.0 mg of PEG-b-PLA were dissolved in 1.0 mL of tert-butanol at 60° C., followed by addition of 1.0 mL of pre-warmed double-distilled water at 60° C. with vigorous mixing. The mixture was allowed to freeze in dry ice/ethanol cooling bath at −70° C. for 2 hours. Lyophilization was then performed on a VirTis AdVantage Pro freeze dryer (SP Scientific, Gardiner, NY) at −20° C. shelf inlet temperature for 72 h at 100 μBar throughout the experiment. The lyophilized cake was then rehydrated with 1.0 mL of MilliQ water or 0.9% saline solution at 60° C., centrifuged, and filtered using a 0.2 μm filter. The drug content in the supernatant was characterized by RP-HPLC. Similar method was employed to prepare o(LLA)₆-GEM or o(LLA)₁₀-GEM loaded PEG-b-PLA micelles.

Conversion of stereocomplex o(L+DLA)₁₀-GEM prodrugs in an CH₃CN/PBS mixture at pH 7.4. A mixture o(LLA)₁₀-GEM (1.0 mg/mL) and o(DLA)₁₀-GEM (1.0 mg/mL) was first dissolved in a 1:1 (v/v) mixture of CH₃CN and PBS (10 mM, pH 7.4), placed in a 1.5 mL Eppendorf tube, and incubated at 37° C. in a temperature adjusted water-bath (GCA Corporation, IL) (see Tam, Y. T. et al., J. Am. Chem. Soc., 2016, 138:8674-8677). 10 μL solution was withdrawn at predetermined time points, and diluted with 90 μL of CH₃CN prior to RP-HPLC analysis. Similarly, conversion of o(LLA)₆-GEM or o(LLA)₁₀-GEM in a 1:1 (v/v) mixture of CH₃CN and PBS (10 mM, pH 7.4) was measured by RP-HPLC. First-order constants were calculated for the degradation kinetics of mixture of o(LLA)₁₀-GEM and o(DLA)₁₀-GEM, o(LLA)₁₀-GEM or o(LLA)₆-GEM. Each experiment was evaluated three times and reported with mean and standard deviation.

Stability of stereocomplex o(L+DLA)₁₀-GEM loaded PEG-b-PLA micelles in water. Stereocomplex o(L+DLA)₁₀-GEM loaded PEG-b-PLA micelles was prepared (2.0 mg/mL) via lyophilization and rehydrated with MilliQ water. The stability of stereocomplex o(L+DLA)₁₀-GEM micelle in water was monitored at room temperature. 10 μL solution was withdrawn at predetermined time points and diluted with 90 μL of CH₃CN prior to RP-HPLC analysis.

Stability of stereocomplex o(L+DLA)₁₀-GEM loaded PEG-b-PLA micelles, mixture of o(LLA)₁₀-GEM and o(DLA)₁₀-GEM, and GEM in rat plasma. The stability of stereocomplex o(L+DLA)₁₀-GEM loaded PEG-b-PLA micelles, mixture of o(LLA)₁₀-GEM and o(DLA)₁₀-GEM, or GEM in rat plasma were determined using heparinized Sprague-Dawley rat plasma (Innovative Research Inc., Novi, MI). Frozen plasma samples were incubated at 37° C. for 5 min before use. Stock solution of stereocomplex o(L+DLA)₁₀-GEM micelles or GEM was prepared in water at 2.0 mg/mL. 100 μL of stereocomplex o(L+DLA)₁₀-GEM micelles or GEM in water was added to 900 μL plasma samples to reach a final concentration of 0.2 mg/mL. For the mixture of o(LLA)₁₀-GEM and o(DLA)₁₀-GEM, stock solution of was prepared in DMSO at 4.0 mg/mL. 10 μL of the mixture in DMSO was added to 990 μL plasma samples to reach a final concentration of 0.04 mg/mL. Samples were incubated at 37° C. in a temperature adjusted water-bath (GCA Corporation, IL). At predetermined time intervals (0, 0.5, 1, 2, 4, and 24 h), 50 μL of plasma samples were withdrawn and diluted with 100 μL of acetonitrile containing 0.1% formic acid. Precipitated samples were centrifuged at 13,000 rpm for 10 min, and the resultant supernatants were analyzed by RP-HPLC.

In vitro release studies. Stereocomplex o(L+DLA)₁₀-GEM, o(LLA)₁₀-GEM, or o(LLA)₆-GEM loaded PEG-b-PLA micelles were diluted to 0.5 mg/mL in 10 mM PBS solution at pH 7.4. A Slide-A-Lyzer™ Dialysis Cassette with MWCO 20K (ThermoFisher, Waltham, MA) was used to load 2.5 mL of diluted micelle solution. Three dialysis cassettes were placed in a 4 L PBS solution (10 mM, pH 7.4) on a Corning Hotplate Stirrer (Corning Inc., Corning, NY) at 37° C. At 0, 1, 2, 3, 6, 9, 24, 48, 72, 120, 168, 216, 312, and 504 h, 100 μL of sample was withdrawn from the dialysis cassette and the cassette was replenished with 100 μL of fresh PBS solution (10 mM, pH 7.4). The external medium was replaced with 4 L of fresh buffer at 2, 6, 24, 72, and 168 h to approximate sink conditions. Drug quantification of stereocomplex o(L+DLA)₁₀-GEM, o(LLA)₁₀-GEM, or o(LLA)₆-GEM in PEG-b-PLA micelles were determined by RP-HPLC. Cumulative percent drug release was calculated, and drug release half—was calculated according to first-order rate equation.

In vitro cytotoxicity studies. The cytotoxicity of GEM, or stereocomplex o(L+DLA)₁₀-GEM micelles at 15% loading against human A549 non-small lung cancer cells and PANC-1 pancreatic cancer cells was investigated by the CellTiter-Blue® Cell Viability Assay (Promega, Madison, WI). A549 cells were seeded into a 96-well plate at a seeding density of 1,500 cells/100 μL/well and cultured in RPMI 1640 medium, or PANC-1 cells were seeded into a 96-well plate at a seeding density of 5,000 cells/100 μL/well and cultured in Dulbecco Modified Eagle Medium (DMEM), at 37° C. in 5% CO₂ incubator for 24 h. GEM, or stereocomplex o(L+DLA)₁₀-GEM micelles at 15% loading in PBS solution (10 mM, pH 7.4) was added into the wells to attain a final concentrations of 1-10,000 nM. Cells cultured with diluted PBS in medium was used as controls. Drug treated cells were placed in an incubator at 5% CO₂ at 37° C. for 72 h. The medium in each well was aspirated, and 100 μL of 20% (v/v) CellTiter-Blue reagent in serum free RPMI 1640 or DMEM was added, followed by incubation at 37° C. in 5% CO₂ atmosphere for 1.5 h. Fluorescence intensity was measured by a SpectraMax M2 plate reader (Molecular Devices, San Jose, CA) with excitation and emission at 560 and 590 nm, respectively. The half maximal inhibitory drug concentration (IC₅₀) was determined by using GraphPad Prism version 5.00 for Windows (GraphPad Software, La Jolla, CA).

In vivo antitumor efficacy studies. All animal experiments were conducted under the protocol approved by Institutional Animal Care and Use Committee in University of Wisconsin-Madison in accordance with institutional and NIH guidance for the Care and Use of Laboratory Animals. 6-8 week-old female athymic nude mice (20-25 g each) were acquired from laboratory animal resources at School of Medicine and Public Health, University of Wisconsin-Madison. Mice were housed in ventilated cages with free access to water and food and acclimated for 1 week prior tumor cell injection. A549 cells (2×10⁶ cells in 100 μL of serum-free RPMI 1640 medium) were harvested from sub-confluent cultures after trypsinization and were injected subcutaneously into the right flank of each mouse. When tumor volume had reached approximately 200-400 mm³, mice were randomly divided into 3 treatment groups (n=3-4/group): GEM at 10 mg/kg, stereocomplex o(L+DLA)₁₀-GEM micelles at 5% loading at 10 mg/kg GEM equivalents, and saline control. Drugs were administered via tail vein for 3 weekly injections. Body weight and tumor volume were monitored over the course of study. Tumor volume was calculated using the formula: V=(a×b²)/2, where V is tumor volume, a is tumor length, b is tumor width.

Statistical analysis. Student t-test at 5% significance level was used for statistical analysis. All data analyses were performed using GraphPad Prism version 5.00 for Windows (GraphPad Software, La Jolla, CA).

### Example 2: Synthesis of o(LLA)ₙ-GEM, o(DLA)ₙ-GEM, or Stereocomplexed o(L+DLA)ₙ-GEM

Monodisperse o(LLA)ₙ or o(DLA)ₙ was synthesized by a ring-opening polymerization (ROP) of either cyclic L-lactide or cyclic D-lactide using benzyl alcohol as initiator and tin(II)-ethylhexanoate (Sn(Oct)₂) as catalyst, followed by fractionation and direct hydrogenation. Coupling of o(LLA)ₙ or o(DLA)ₙ with GEM at the 4-(N)-position via amide linkage was mediated by 1-ethyl-3-(3-dimethylaminopropyl)carbodiimide (EDCI) and N-hydroxysuccinimide (NHS), resulting in o(LLA)ₙ-GEM and o(DLA)ₙ-GEM prodrugs (n=6 or 10) (FIG. 2). The chemical structures were supported by ¹H NMR spectroscopy and electrospray mass spectroscopy analysis.

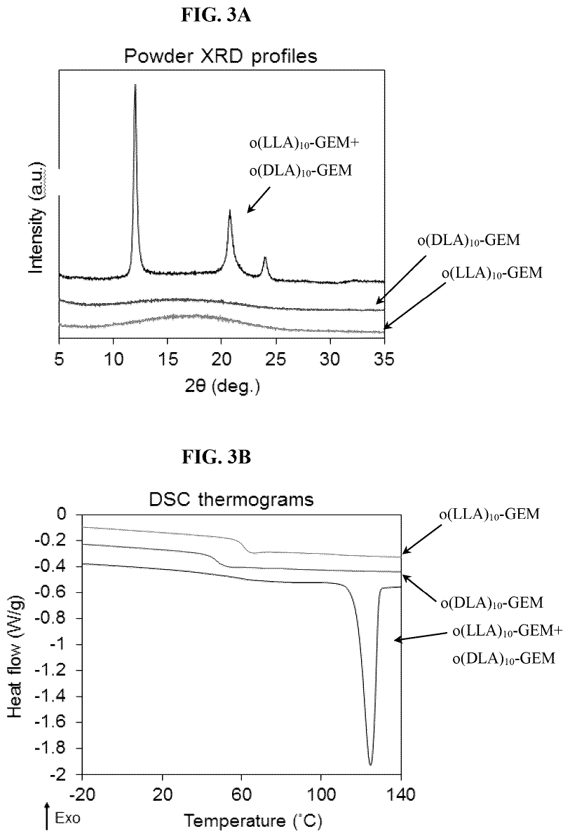

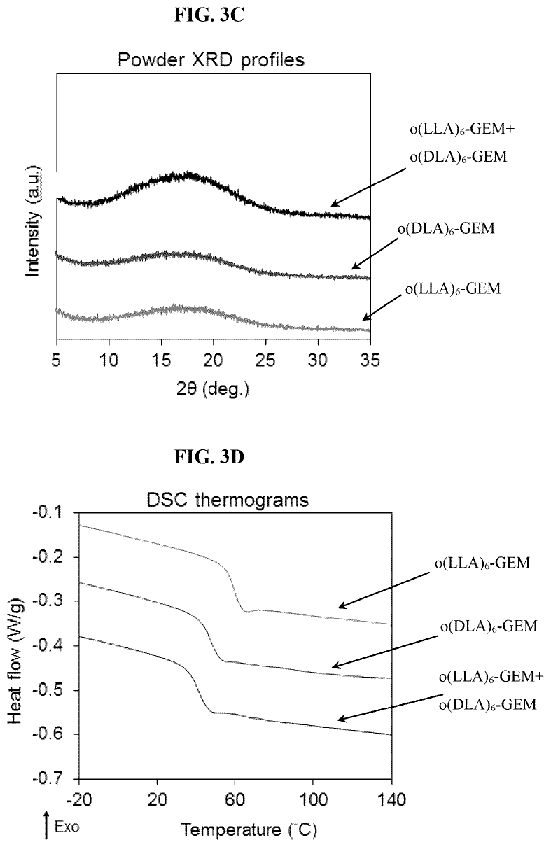

To explore the stereocomplex formation of o(LLA)ₙ-GEM and o(DLA)ₙ-GEM, both prodrugs were mixed in an equal mass ratio in a glass vial before dissolving in acetonitrile (CH₃CN). The resulting solution was then placed in a vacuum oven at 70° C. for 12 h to allow complete solvent removal. Powder X-ray diffraction (PXRD) (FIGS. 3A and 3C) and differential scanning calorimetry (DSC) (FIGS. 3B and 3D) were used to confirm the formation of crystalline stereocomplex through the identification of different diffraction patterns and melting transition (Tm) as compared to each enantiomeric form of the prodrug. When n=10, both o(LLA)₁₀-GEM and o(DLA)₁₀-GEM are amorphous, as evidenced by the broad diffraction patterns and the absence of a DSC melting endotherm. By contrast, a 1:1 mixture of o(LLA)₁₀-GEM and o(DLA)₁₀-GEM shows three distinct crystalline peaks at 12.0°, 20.8°, and 23.9°; which is the unique diffraction signature of the formation of crystalline PLA stereocomplex (see Ikada, Y. et al., Macromolecules, 1987, 20:904-906). The presence of the crystalline stereocomplex was further proven by a melting endotherm at 117.8° C. Interestingly, when n=6, no crystalline stereocomplex can be formed after blending equal mass ratio of o(LLA)₆-GEM and o(DLA)₆-GEM, as evidenced by a broad diffraction peak and the absence the melting endotherm (FIGS. 3C and 3D).

### Example 3: Characterization of PEG-b-PLA Micelles Containing o(LLA)ₙ-GEM, o(DLA)ₙ-GEM, or Stereocomplexed o(L+DLA)ₙ-GEM

The incorporation of o(LLA)₆-GEM, o(LLA)₁₀-GEM, or stereocomplex o(L+DLA)₁₀-GEM in PEG-b-PLA micelles was achieved by lyophilization from a tert-butanol-water mixture. As shown in Table 1, both o(LLA)₆-GEM and o(LLA)₁₀-GEM were successfully incorporated in PEG-b-PLA, forming micelles with an average hydrodynamic diameter at ca. 30 nm and ca. 7% drug loading.

TABLE 1
Physicochemical characterizations of o(LLA)₆-GEM, o(LLA)₁₀-GEM, and stereocomplex
o(L + DLA)₁₀-GEM loaded PEG-b-PLA micelles (mean ± SD, n = 3).

| | Particle | | Loading | Loading | Stability |
|---|---|---|---|---|---|
| Drug | size (nm) | PDI | efficiency (%) | content (%) | (h)^{d)} |
| o(LLA)₆-GEM^{a)} | 27.4 ± 0.3 | 0.23 ± 0.01 | 67.5 ± 11.3 | 6.3 ± 1.1 | <24 |
| o(LLA)₁₀-GEM^{a)} | 29.4 ± 0.1 | 0.13 ± 0.03 | 82.1 ± 11.4 | 7.6 ± 1.1 | <1 |
| o(L + DLA)₁₀-GEM^{b)} | 139.5 ± 24.9 | 0.35 ± 0.08 | 56.7 ± 4.3 | 5.4 ± 0.4 | >168 |
| o(L + DLA)₁₀-GEM^{c)} | 202.3 ± 19.0 | 0.33 ± 0.07 | 90.3 ± 4.4 | 15.3 ± 0.9 | >168 |

^{a)}1.0 mg of o(LLA)₆-GEM or o(LLA)₁₀-GEM and 10.0 mg of PEG₄₀₀₀-b-PLA₂₂₀₀ were used.
^{b)}1.0 mg of o(LLA)₁₀-GEM, 1.0 mg of o(DLA)₁₀-GEM and 20.0 mg of PEG₄₀₀₀-b-PLA₂₂₀₀ were used.
^{c)}1.0 mg of o(LLA)₁₀-GEM, 1.0 mg of o(DLA)₁₀-GEM and 10.0 mg of PEG₄₀₀₀-b-PLA₂₂₀₀ were used.
^{d)}Formulations were dispersed in MilliQ water at 25° C.

Without wishing to be bound by theory, it is believed that introducing o(LLA)ₙ to GEM as a pro-moiety can enhance the compatibility of GEM in the PLA core of PEG-b-PLA micelles. However, loading of o(LLA)₆-GEM or o(LLA)₁₀-GEM within micelles did not show improve physical stability, precipitating in <24 hours or <1 hour, respectively. In contrast, PEG-b-PLA micelles containing stereocomplex o(L+DLA)₁₀-GEM demonstrated superior physical stability, with no substantial change in particle size and no drug precipitation >168 hours. Interestingly, the incorporation of stereocomplex o(L+DLA)₁₀-GEM in PEG-b-PLA micelles at 5.4% and 15.3% increased the hydrodynamic diameter from 30 nm to ca. 140 nm and ca. 200 nm, respectively, compared to their non-complexed counterparts.

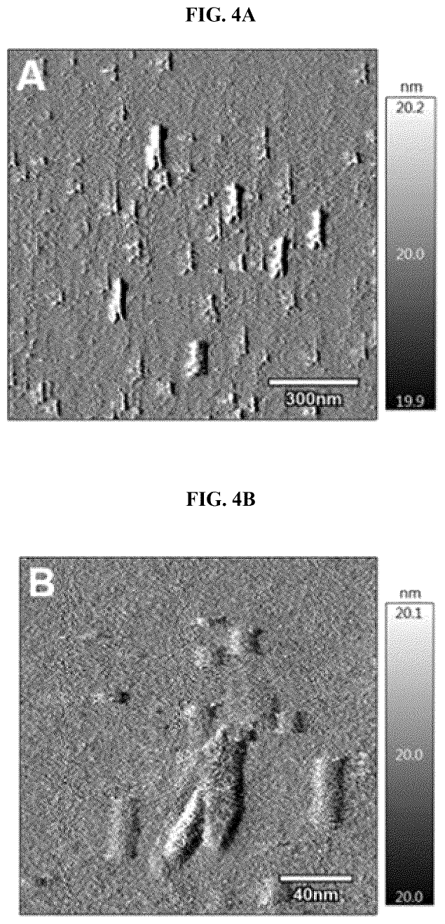

The morphology of o(L+DLA)₁₀-GEM micelles was further investigated using atomic force microscopy (AFM). FIGS. 4A and 4B show the unexpected, elongated structure of stereocomplexed micelles. A distinct difference of this system, compared to other micelles containing diblock copolymers, is that stereocomplex o(L+DLA)₁₀-GEM prodrugs are loaded inside the core of PEG-b-PLA micelles, instead of direct stereocomplexation between the core blocks of the copolymer.

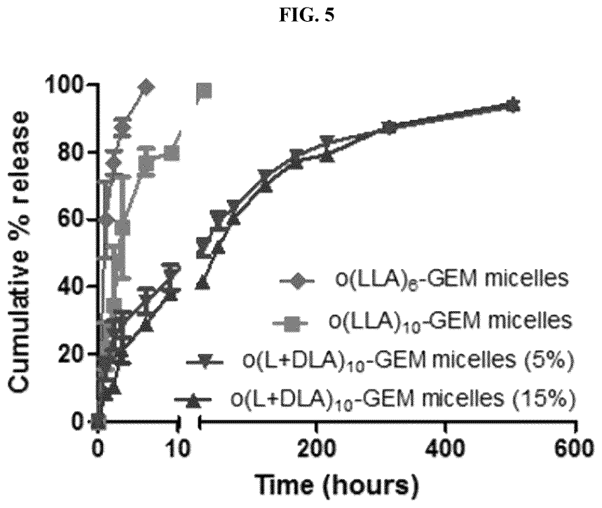

FIG. 5 demonstrates the release of o(LLA)₆-GEM and o(LLA)₁₀-GEM from PEG-b-PLA micelles were relatively rapid in vitro, with t_{1/2} at ca. 0.8 hours and ca. 4 hours, respectively. It may be attributed to the hydrophilic nature of GEM, resulting in reduced partition of the drug for the hydrophobic micelle core of PEG-b-PLA micelles, and therefore faster drug release. By contrast, stereocomplex o(L+DLA)₁₀-GEM was gradually released from PEG-b-PLA micelles at 5.4% and 15.3%, both with t_{1/2} at ca. 60 hours in the first 3 days, and followed by a much slower release rate thereafter throughout three weeks (FIG. 5). These data demonstrate that stereocomplex o(L+DLA)₁₀-GEM was stably loaded in PEG-b-PLA micelles with an unexpected cylindrical morphology, demonstrating superior physical stability and sustained drug release.

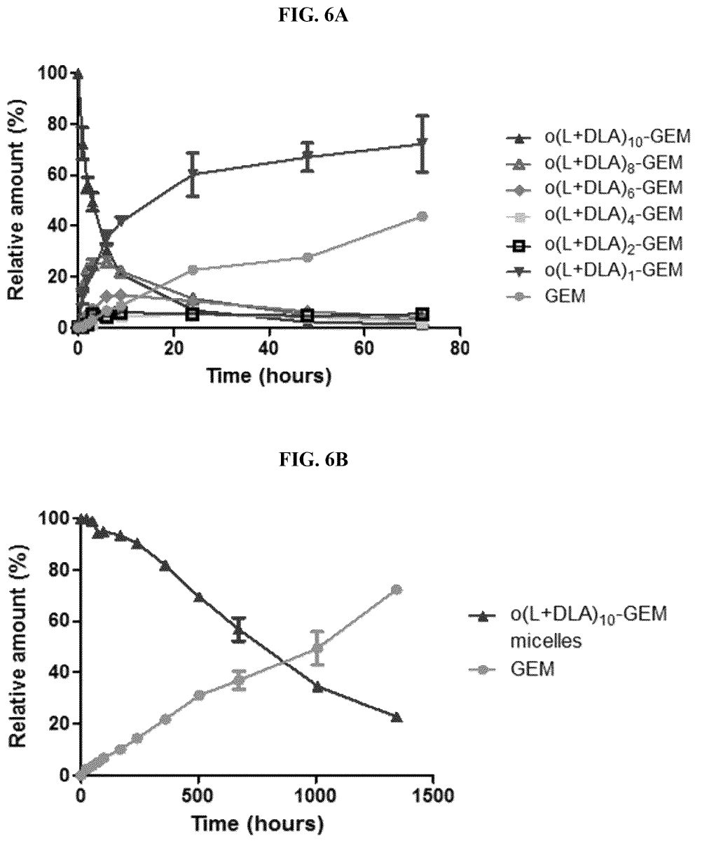

Degradation of oligolactic acid conjugates is driven by an intramolecular backbiting reaction of the terminal hydroxyl group on the penultimate ester bond of o(LA)ₙ in a mixture of acetonitrile and phosphate buffered saline (1:1 v/v CH₃CN/PBS), cleaving lactoyl lactate in a stepwise chain end scission. o(L+DLA)₁₀-GEM backbiting kinetics was expected to be slower due to the strong interaction of PLA stereocomplex however, conversion of o(L+DLA)₁₀-GEM (λₘₐₓ=300 nm) was found to have a similar backbiting kinetics in 1:1 v/v CH₃CN/PBS, with t_{1/2} at ca. 6.6 hours versus ca. 7.3 hours of o(LA)₈-PTX (FIG. 6A), see Tam, Y. T. et al., J. Am. Chem. Soc., 2016, 138:8674-8677). On the contrary, degradation of o(L+DLA)₁₀-GEM in PEG-b-PLA micelles was remarkably slow, with t_{1/2} at ca. 630 hours (FIG. 6B). The superior stability of o(L+DLA)₁₀-GEM in PEG-b-PLA micelles could be ascribed to the stable stereocomplexation between o(LLA)₁₀ and o(DLA)₁₀ pro-moieties in the PLA core of PEG-b-PLA micelles, which potentially inhibited backbiting. However, when o(L+DLA)₁₀-GEM was dissolved in the CH₃CN/PBS mixture, no stereocomplexation was formed due to complete solubilization of o(L+DLA)₁₀-GEM, and therefore backbiting conversion followed.

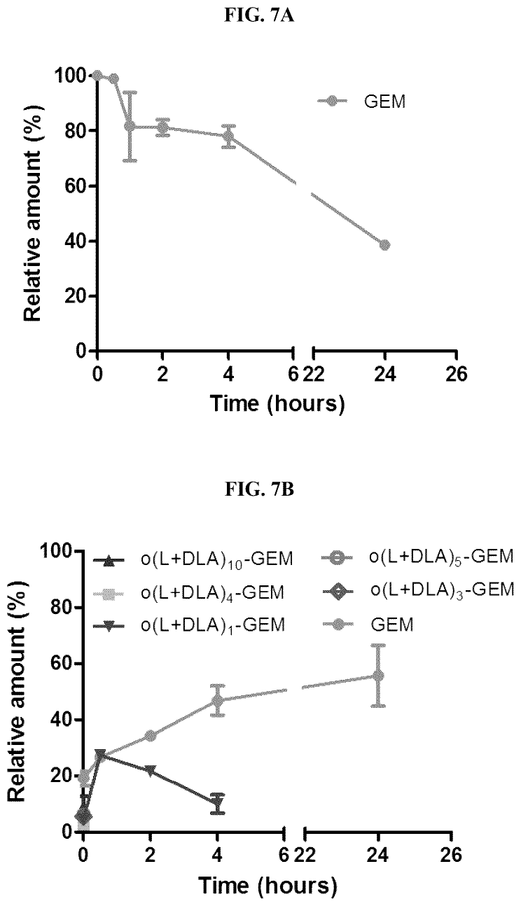

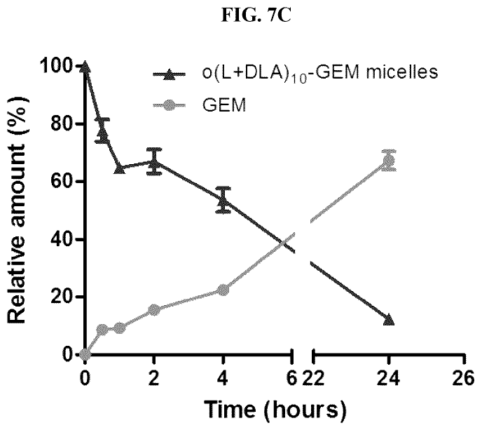

GEM is susceptible to deamination in plasma by metabolic enzymes like cytidine deaminase, rendering it pharmacologically inactive. Without wishing to be bound by theory, it is believed that introducing the attachment of a pro-moiety at the 4-(N)-position of GEM will obscure the deamination site, and therefore improve metabolic stability of GEM. Consequently, the metabolic stability of o(L+DLA)₁₀-GEM in PEG-b-PLA micelles and o(L+DLA)₁₀-GEM in comparison to GEM were investigated in rat plasma. FIG. 7A demonstrates that GEM was not stable in plasma, with less than 50% of intact GEM detected after 24 hours incubation in plasma at 37° C. Surprisingly, the degradation of o(L+DLA)₁₀-GEM was extremely rapid in plasma, generating random distribution of degradation species and ca. 20% GEM during the first sampling time point, suggesting fragile amide linkage and contribution of esterases in addition to backbiting (FIG. 7B). When o(L+DLA)₁₀-GEM was loaded in PEG-b-PLA micelles, o(L+DLA)₁₀-GEM gained extra stability through stereocomplexation, slowly generating GEM as the only major degradation species in plasma over 24 hours (FIG. 7C). Notably, no degradation intermediates were generated throughout the study, indicating stable stereocomplexation of o(L+DLA)₁₀-GEM in PEG-b-PLA micelles could potentially prevent esterase degradation of o(LLA)ₙ and o(DLA)ₙ.

### Example 4: In Vitro and In Vivo Anticancer Activity of Stereocomplexed o(L+DLA)ₙ-GEM Micelles

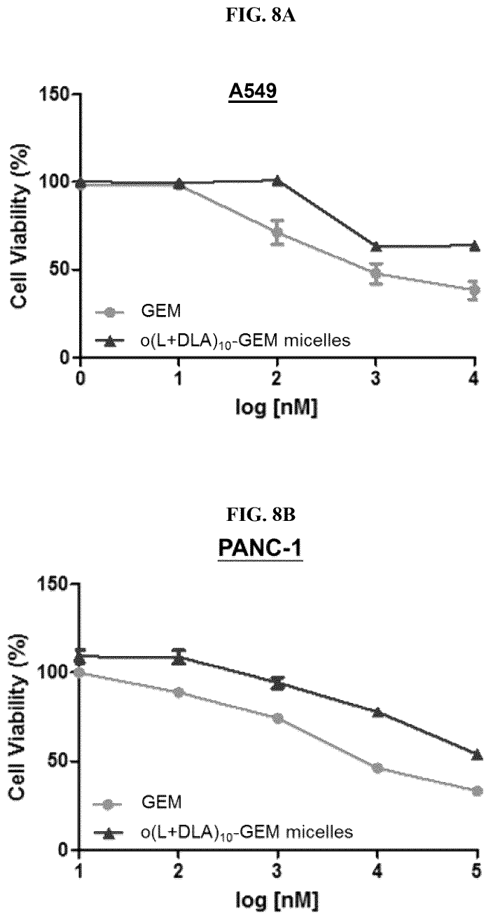

The bioactivity of o(L+DLA)₁₀-GEM micelles was evaluated by in vitro cell viability assay to test the half maximal inhibitory concentration (IC₅₀) in comparison to GEM. Since GEM was used as the first-line treatment for non-small cell lung cancer (NSCLC) and pancreatic cancer, human A549 NSCLC cell line and PANC-1 pancreatic cancer cell line were investigated using a CellTiter-Blue assay. GEM had a relatively low IC₅₀ value against A549 (FIG. 8A) and PANC-1 (FIG. 8B) cells, at ca. 1.1 μM and ca. 9.2 μM, respectively. In contrast, o(L+DLA)₁₀-GEM micelles were less cytotoxic than GEM in A549 (FIG. 8A) and PANC-1 (FIG. 8B) cells after 72 hours incubation, with an IC₅₀ value of ca. 12.7 μM and ca. 97.9 μM, respectively. The in vitro cytotoxicity data provided further supportive evidence of high stability of o(L+DLA)₁₀-GEM stereocomplex in PEG-b-PLA micelles and the remarkably slow prodrug release from micelles, which led to decreased cytotoxicity.

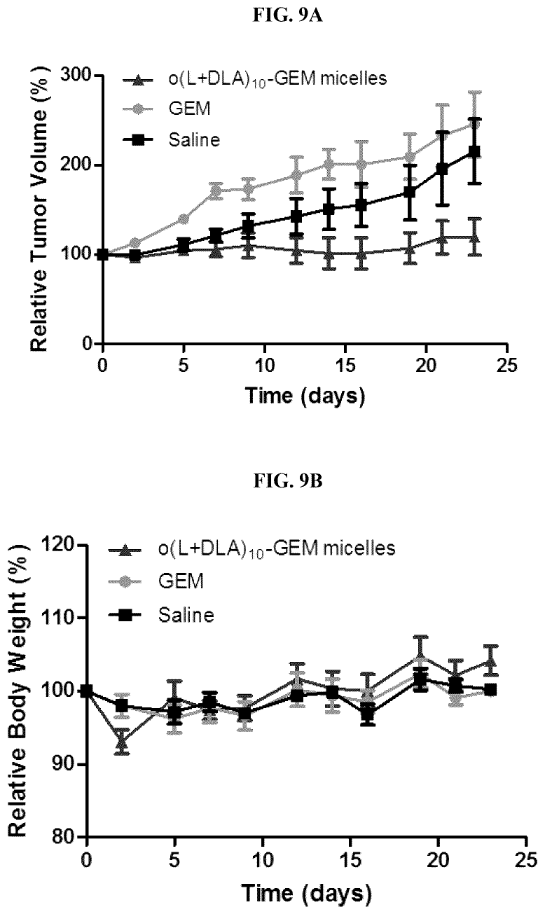

To verify the antitumor efficacy of o(L+DLA)₁₀-GEM micelles in vivo, mice bearing subcutaneous A549 xenografts were treated with normal saline, GEM, or o(L+DLA)₁₀-GEM micelles via 3-weekly I.V. injections at 10 mg/kg equivalent of GEM. Notably, administration of o(L+DLA)₁₀-GEM micelles showed significant tumor growth inhibition compared to GEM or saline control (FIG. 9A). Interestingly, tumors in mice that were treated with GEM grew at a faster rate than saline control, indicating GEM was not effective (FIG. 9A). A slight decrease of body weight was recorded after the first dose of o(L+DLA)₁₀-GEM micelles, but no significant difference was observed among all the treatment groups (FIG. 9B). Delivery of GEM at 10 mg/kg is generally ineffective against subcutaneous tumors in mice. Generally, 100 mg/kg of GEM is required to inhibit tumor growth in mice bearing subcutaneous tumors due to its metabolic instability. Reported maximum tolerated doses (MTD) of the squalenoyl GEM nanoassemblies in mice (20 mg/kg) was 5-fold lower than that of GEM (100 mg/kg). This study's in vivo efficacy results are supported by these findings. Although a relatively low dose of GEM was used (10 mg/kg), o(L+DLA)₁₀-GEM micelles displayed potent antitumor effect in vivo, suggesting stable o(L+DLA)₁₀-GEM stereocomplexation in PEG-b-PLA micelles could effectively deliver GEM to tumor site for drug action.

### EQUIVALENTS

While certain embodiments have been illustrated and described, a person with ordinary skill in the art, after reading the foregoing specification, can effect changes, substitutions of equivalents and other types of alterations to the conjugates and micelles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1.-23. (canceled)

24. A composition comprising water and micelles, wherein the micelles comprise a poly(ethylene glycol)-block-polylactic acid (PEG-b-PLA) copolymer, and are loaded with a stereocomplex of a 4(N)-oligo-L-lactic acid conjugate of gemcitabine or a gemcitabine derivative with a 4(N)-oligo-D-lactic acid conjugate of gemcitabine or a gemcitabine derivative;
wherein:
the oligo-L-lactic acid and the oligo-D-lactic acid in each conjugate comprises 7 to 20 lactic acid subunits; and
the oligo-L-lactic acid or the oligo-D-lactic acid is attached through an amide linkage to the nitrogen of the 4-amino of the gemcitabine or gemcitabine derivative.

25. The composition of claim 24, wherein the molar ratio of the 4(N)-oligo-L-lactic acid conjugate of gemcitabine or a gemcitabine derivative to the 4(N)-oligo-D-lactic acid conjugate of gemcitabine or a gemcitabine derivative ranges from about 2:1 to about 1:2.

26. The composition of claim 24, wherein the molar ratio of the 4(N)-oligo-L-lactic acid conjugate of gemcitabine or a gemcitabine derivative to the 4(N)-oligo-D-lactic acid conjugate of gemcitabine or a gemcitabine derivative is about 1:1.

27. The composition of claim 24, wherein the 4(N)-oligo-L-lactic acid conjugate and the 4(N)-oligo-D-lactic acid conjugate are conjugates of gemcitabine.

28. The composition of claim 24, wherein the loading of the stereocomplex is from about 1 wt % to about 50 wt %, with respect to the mass of the micelles.

29. The composition of claim 24, wherein the concentration of the stereocomplex is from about 0.6 mg/mL to about 40 mg/mL, with respect to the volume of the water in the composition.

30. The composition of claim 24, wherein the molecular weight of the poly(ethylene glycol) block of the PEG-b-PLA copolymer is about 1,000 g/mol to about 35,000 g/mol; the molecular weight of the poly(lactic acid) block of the PEG-b-PLA copolymer is about 1,000 g/mol to about 15,000 g/mol; or a combination thereof.

31. A method of making the composition of claim 24, comprising freeze-drying a t-butanol/water solution comprising a stereocomplex of a 4(N)-oligo-L-lactic acid conjugate of gemcitabine or a gemcitabine derivative with a 4(N)-oligo-D-lactic acid conjugate of gemcitabine or a gemcitabine derivative;
wherein:
the oligo-L-lactic acid or the oligo-D-lactic acid in each conjugate comprises 7 to 20 lactic acid subunits; and
the oligo-L-lactic acid or the oligo-D-lactic acid is attached through an amide linkage to the nitrogen of the 4-amino of the gemcitabine or gemcitabine derivative.

32. A method of inhibiting or killing cancer cells sensitive to gemcitabine or a gemcitabine derivative comprising contacting the cancer cells with an effective inhibitory or lethal amount of a composition of claim 24.

33. The method of claim 32, wherein the contacting occurs in vitro.

34. A method of treating cancer in a subject in need thereof, comprising administering to the subject an effective amount of the composition of claim 24, wherein the cancer is sensitive to gemcitabine or a gemcitabine derivative.

35. The method of claim 34, wherein the cancer is selected from the group consisting of ovarian cancer, leukemia, angiosarcoma, breast cancer, colorectal cancer, prostate cancer, lung cancer, pancreatic cancer, cholangiocarcinoma, brain cancer (such as gliomas), adenocarcinomas, hepatomas, and biliary tract cancer.

36. The method of claim 34, wherein the cancer is selected from the group consisting of: ovarian cancer, breast cancer, lung cancer, pancreatic cancer, cholangiocarcinoma, and biliary tract cancer.

37. The method of claim 34, wherein the cancer is lung cancer or pancreatic cancer.

38. The method of claim 34, wherein the composition is administered topically, intranasally, systemically, intravenously, subcutaneously, intraperitoneally, intradermally, intraocularly, iontophoretically, transmucosally, or intramuscularly.
